(19) United States
(12) Patent Application Publication
(10) Pub. No.: US 2011/0111386 A1
(43) Pub. Date: May 12, 2011

(54) CERVICAL CELL COLLECTION METHOD

(75) Inventors: Runi Rogers, Klokkarstua (NO); Hanne Skomedal, Klokkarstua (NO)

(73) Assignee: NORCHIP AS, Klokkarstua (NO)

(21) Appl. No.: 12/616,222

(22) Filed: Nov. 11, 2009

(51) Int. Cl. C12Q 1/70; C12Q 1/68; A61B 10/00

(52) U.S. Cl. 435/5; 435/6; 600/562

(57) ABSTRACT

The present invention relates to sample collection and preservation. More specifically, the invention provides methods for the collection and preservation of at least one nucleic acid molecule in a test sample comprising one or more cells or tissues obtained from the cervix of a subject.

### FIELD OF THE INVENTION

The present invention relates to sample collection and preservation. More specifically, the invention provides methods for the collection and preservation of at least one nucleic acid molecule in a test sample comprising one or more cells or tissues obtained from the cervix of a subject.

### BACKGROUND TO THE INVENTION

Vaginal and cervical cell health may be adversely affected by a number of different factors including vaginitis, yeast infection, cervical dysplasia and cervical cancer. In order to facilitate monitoring of cervical health, routine cervical screening programs have been implemented in several countries worldwide.

As an example, the number of new cervical cancer cases diagnosed annually ranges from 5 to 42 per 100,000 of the population (2002; WHO). The development of cervical cancer is closely associated with human papillomavirus (HPV) infection. HPV infection can induce cervical cell transformation and the maintenance of neoplastic or dysplastic cell phenotypes. Of the ˜130 HPV types that have been identified, infection with types 16, 18, 31, 33, 35, 52, 58 and 67 is considered to increase the risk of developing cervical cancer. Cervical screening programs are used to detect changes in cervical cell morphology to allow early detection and treatment of pre-malignant and malignant cells. Such programs traditionally require a trained medical practitioner (for example a gynaecologist, general practitioner or nurse) to collect a cervical specimen. Cells are collected by scraping the outer opening of the cervix with a spatula, an endocervical brush or a swab (the “papanicolaou smear test”, also referred to herein as a “Pap smear”). Typically, the removed cells are placed on a microscope slide with a fixative. The sample is then transported to a remote site for histopathological analysis using light microscopy.

Changes in cellular characteristics other than morphology may also indicate a change in the health status of a cell. In particular, infection or transformation of cervical cells may affect mRNA production, mRNA stability and/or protein expression. By way of example, HPV infection may result in continuous E6/E7 gene expression in infected cells, which, in turn, induces cellular transformation of infected cells into malignant cells. Monitoring E6/E7 expression in such cells therefore facilitates early diagnosis and possible prevention of spread of disease. E6/E7 mRNA expression levels may be studied using several different methods, including Northern blotting, nuclease protection assays RT PCR assays, transcription mediated amplification (TMA) or the PreTect™ HPV-Proofer assay.

One method of obtaining cervical cells for subsequent analysis involves the use of a tampon. Delany et al (J Acquir Immune Defic Syndr, 2008, Dec. 1; 49(4):406-9) and Webber et al (AIDS, 20001, July 27; 15(11):1417-20) have shown that samples obtained by self collection of cervical cells using a tampon may be used in the detection of HIV-1/HSV-2 infection. In addition, Harper et al (Am J Obstet Gynecol, March 2002) have shown that tampons may be used for self sampling of cervical cells for obtaining DNA for subsequent HPV testing in subjects with abnormal cervical cytologic conditions.

### SUMMARY OF THE INVENTION

Typically, 4 to 9% of papanicolaou smear test results reported in the UK show an abnormal phenotype (NHS information centre). However, most of these results are only mildly abnormal; the cervical dysplasia will most likely spontaneously regress without developing into cervical cancer. Identifying and reporting on such abnormalities may be disadvantageous, since it is likely to induce unnecessary patient stress and uncertainty.

An alternative means of detecting HPV infection is provided by the PreTect™ HPV-Proofer test, which is a multiplex nucleic acid sequence based amplification (NASBA) assay that exclusively detects HPV type specific E6/E7 mRNA from carcinogenic HPV types (Molden et al., Journal of Virological Methods, 142 (1-2): 204-12, 2007). It is a useful tool in the detection of oncogene activity related to the development of cervical cancer. In addition, the method is relatively sensitive; the presence of one affected cell within a test sample may be sufficient for positive identification of E6/E7 mRNA expression. However, to function optimally, the PreTect™ HPV-Proofer assay requires mRNA of a certain quality. To ensure assay accuracy and reproducibility, collection and analysis of the cervical sample must be performed before mRNA degradation has taken place. This is difficult, since RNase enzymes are released upon cell lysis and are also present within the environment. In light of the above issues, standard cervical screening programs have traditionally retained loyalty to the papanicolaou smear test.

The conventional methods for collection and preservation of cervical cells for subsequent analysis require a trained medical practitioner to correctly isolate the cells and ensure that the sample is suitable for histopathological analysis. Unfortunately, the physical examination required to collect the cells from a patient may not be acceptable to certain individuals with particular ethical, religious or personal beliefs. Moreover, trained medical practitioners may not be easily accessible to all individuals that would benefit from cervical screening. This may limit the usefulness of a cervical screening program significantly.

The present invention has been conceived in light of the significant number of disadvantages and inconveniences that exist in the screening technologies currently available.

The present invention thus provides methods for the collection and preservation of at least one nucleic acid molecule in a test sample comprising one or more cells or tissues obtained from the cervix of a subject.

In a first aspect, the invention provides a method of collecting one or more cells or tissues from the cervix of a subject and preserving said cells or tissues in a manner which permits recovery of at least one nucleic acid molecule in a test sample, the method comprising the steps of contacting the cervix of a subject with a collection means of absorbent material to collect the test sample and bringing the test sample into contact with a nucleic acid preservation solution. The method is designed for home testing and thus the subject contacts their cervix with a collection means of absorbent material to collect the test sample.

Optionally, the preservation of said cells or tissues is performed at ambient or near ambient temperature.

Preferably, the collection means of absorbent material of the methods of the invention is a disposable absorbent product of cotton and/or rayon staple wadding suitable for insertion into the vaginal cavity of the subject. Advantageously, the collection means of absorbent material is suitable for insertion into the vaginal cavity by the user, thereby allowing self-sampling. Optionally, the collection means of absorbent material further comprises a removal cord or pull string. Most advantageously, the collection means of absorbent material is a menstruation tampon. Advantageously, the menstruation tampon further comprises an applicator.

In one embodiment, the collection means of absorbent material of the method is retained within the vaginal cavity of the subject for a sufficient length of time to collect a test sample suitable for subsequent nucleic acid extraction. Preferably, the collection means of absorbent material is retained within the vaginal cavity of the subject for approximately at least one hour. Advantageously, subsequent nucleic acid extraction comprises RNA extraction.

In a related aspect, the invention provides a method of collecting one or more cells or tissues from the cervix of a subject and preserving said cells or tissues in a manner which permits recovery of at least one nucleic acid molecule in a test sample, the method comprising the steps of contacting the cervix of a subject with a collection means of absorbent material to collect the test sample and bringing the test sample into contact with a nucleic acid preservation solution, wherein bringing the test sample into contact with a nucleic acid preservation solution comprises placing the collection means of absorbent material into a test sample receiving vessel with a body portion.

Preferably, the test sample receiving vessel further comprises a lid portion to fit the body portion, wherein the lid portion and body portion in combination provide a sealable vessel which can (fully) accommodate the nucleic acid preservation solution and the collection means of absorbent material. Optionally, the test sample receiving vessel is a 50 ml sealable container.

In one embodiment of this aspect, the test sample receiving vessel of the method contains a nucleic acid preservation solution. Preferably, the nucleic acid preservation solution of the method comprises methanol at a concentration in the range of at least 60% (v/v).

In a further embodiment of the methods of the invention, the test sample is immersed in the nucleic acid preservation solution when it is brought into contact with the nucleic acid preservation solution.

In certain embodiments of this aspect, the method provides for collection of a test sample suitable for RNA extraction. The method may be used to collect a test sample that is suitable for subsequent detection of a cervical malignancy in a subject.

In each embodiment of the methods of the invention, the subject may be an animal or a human.

The subject may be asymptomatic, typically an asymptomatic human female. Included within the category of “asymptomatic subjects” are subjects who have not previously been tested for cervical pre-cancer, a pre-disposition to cervical cancer or an incidence of cervical malignancy. This includes, for example, subjects who have never been tested by Pap smear or liquid-based cytology, but do not show any outward symptoms of cervical malignancy.

Also included within the category of “asymptomatic” subjects are subjects who appear normal based on Pap smear and/or liquid-based cytology. Self-sampling of cervical cells, and subsequent testing for HPV DNA and/or mRNA, may be especially useful for individuals who do not have access to a primary cervical screening program, or for other reasons have never been enrolled in primary screening, and also for individuals who desire a test for HPV DNA and/or mRNA voluntarily, perhaps as an adjunct to a cytology-based test, especially of HPV-testing is not routinely available as part of the screening program (for primary screening).

In other embodiments, the subject may have cervical pre-cancer. Cervical pre-cancer may be defined as a lesion from which a malignant tumor is presumed to develop in a significant number of instances and that may or may not be recognizable clinically or by microscopic changes in the affected tissue. Included within the definition of cervical precancer are ASCUS and also CIN1, CIN2 or CIN3 cervical lesions, all of which are accepted terms of art. Self-sampling of cervical cells, and subsequent testing for HPV DNA and/or mRNA, may be useful for individuals who have been diagnosed with some form of cervical pre-cancer, such as ASCUS or CIN1, CIN2 or CIN3 cervical lesions, but have not been tested for HPV. Such individuals may be tested for HPV as a form of triage, e.g. as part of an organised screening program, or may elect voluntarily to take an HPV test if this is not routinely available under the screening program.

Alternatively, the subject may have been treated for cervical pre-cancer or a previous cervical malignancy and is no longer suspected of having the disease. Within the context of this invention, cervical malignancy or cervical cancer may be defined as a cervical tumour that is malignant (i.e. cancerous) that can invade and destroy nearby tissue and that may spread (metastasize) to other parts of the body. Cervical pre-cancer may be further defined as an asymptomatic state that is likely to develop into cervical cancer in the absence of treatment and/or intervention. In one embodiment, subjects have no previous signs of cervical cancer.

The invention also provides a method for preserving a test sample comprising one or more cells or tissues that have been collected from the cervix of a subject by contacting a collection means of absorbent material with the cervix, wherein the preservation is performed in a manner which permits recovery of at least one nucleic acid molecule in a test sample, the method comprising the step of bringing the test sample into contact with a nucleic acid preservation solution.

The methods of the invention may further comprise transporting the test sample from the site of collection from the subject to a site of clinical microbiological testing. In one embodiment, the methods of the invention are performed at ambient temperature as understood by the person skilled in the art. Preferably, the methods of the invention may tolerate variable temperatures. Thus, the methods of the invention may be performed under climatic conditions that are not controlled. More specifically, a preferred temperature range is between −4° C. to 37° C. Most preferably, the temperature range may be between 15° C. to 25° C. By way of example, one or more of the following steps may be performed at ambient temperature (preferably between −4° C. to 37° C., and most preferably between 15° C. to 25° C.); bringing the test sample into contact with the nucleic acid preservation solution, preserving and storing the test sample in the nucleic acid preservation solution and/or transporting the test sample from the site of collection from the subject to a site of clinical microbiological testing.

Preferably, the test sample is preserved and stored within the nucleic acid preservation solution at ambient temperature. Most preferably, preservation and storage of the test sample at ambient temperature does not adversely affect the nucleic acid molecule(s) present within the test sample and permits recovery of at least one or more nucleic acid molecule(s) suitable for subsequent analysis. Advantageously, storage of the test sample at ambient temperature ensures that a test sample may be collected by the user without the need for specialised conditions. For example, the user may collect and preserve the test sample within the comfort of their home. Conveniently, storage at ambient temperature also allows the test sample to be transported from the site of collection from the subject to a site of clinical microbiological testing without requiring any special conditions. For example, the test sample does not require packing on ice.

In one embodiment of the invention, the methods further comprise monitoring and/or analysis of the test sample at the site of clinical microbiological testing. Optionally, a time delay of occurs between bringing the test sample into contact with the nucleic acid preservation solution and the monitoring and/or analysis of the test sample. Optionally, the time delay is up to one week, up to four weeks, up to eight weeks or more than one week. Preferably, once the test sample is at a site of clinical microbiological testing, it is analysed using Nucleic Acid Sequence Based Amplification (NASBA).

In one embodiment of this aspect, the invention relates to a method for collecting one or more cells or tissues from the cervix of a subject and preserving said cells or tissues in a manner that permits recovery of at least one nucleic acid molecule in the test sample, wherein the nucleic acid molecule comprises, consists essentially of or consists of RNA. Optionally, the RNA comprises, consists essentially of or consists of mRNA. Optionally, the test sample is analysed for the presence of human papillomavirus (HPV) mRNA. For example, the test sample may be analysed for the presence of E6/E7 mRNA. In one embodiment, the test sample is analysed for the presence of HPV mRNA derived from carcinogenic HPV types. Preferably, the presence of mRNA is detected using a multiplex Nucleic Acid Sequence Based Amplification (NASBA) assay. For example, the NASBA assay may exclusively detect HPV type specific E6/E7 mRNA from carcinogenic HPV types (for example the PreTect™ HPV proofer assay).

The methods of the invention may also further comprise the step of informing the subject that they have cervical pre-cancer, a pre-disposition to cervical cancer or an incidence of cervical malignancy. Optionally, the methods of the invention comprise the step of detecting cervical pre-cancer, a pre-disposition to cervical cancer or an incidence of cervical malignancy in a subject.

In one embodiment, a method is provided for detecting cervical pre-cancer, a pre-disposition to cervical cancer or an incidence of cervical malignancy in a subject, wherein the method comprises supplying the subject with a sampling kit suitable for the collection of one or more cells or tissues from their cervix. The subject may then collect one or more cells or tissues from their cervix and send the test sample to a site of clinical microbiological testing. Subsequently, monitoring and/or analysis of the test sample at the site of clinical microbiological testing may be carried out in order to detect cervical pre-cancer, a pre-disposition to cervical cancer or an incidence of cervical malignancy. Finally, the results of the monitoring and/or analysis step may be transmitted to the subject or a healthcare professional. The results may be transmitted by methods including, but not limited to, email, telephone, facsimile or text message.

Optionally, the subject has cervical pre-cancer. For example, the method may be used to screen asymptomatic subjects for cervical pre-cancer, a predisposition to a cervical malignancy or an incidence of a cervical malignancy. Such subjects may, for example, decide to perform the method of the invention to determine whether or not they have cervical pre-cancer, a predisposition to a cervical malignancy or an incidence of a cervical malignancy, irrespective of whether or not they are currently or have been part of a routine cervical screening program.

Optionally, the subject collects one or more cells or tissues from their cervix and sends the test sample to a site of clinical microbiological testing at ambient temperature.

The components used in the methods of the invention may typically be retained as a (sampling) kit or as individual, separable components prior to collection of the test sample. Optionally, the nucleic acid preservation solution is retained within the test sample receiving vessel prior to collection of the test sample. It is not essential that the components are sterile prior to collection of the test sample since the down stream analysis methods are (relatively) specific (i.e. most contaminants derived from the user would not necessarily affect down stream analysis). For example, the PreTect™ HPV Proofer test is specific for mRNA from 5 different HPV types and will amplify these mRNAs only, even in the presence of other cellular contaminants. However, preferably, each component is hygienically clean prior to collection of the test sample. The term ‘hygienically clean’ means that the components are suitable for their intended use within the methods of the invention. For example, a hygienically clean collection means of absorbent material is suitable for insertion into the vaginal cavity of the subject. In a preferred embodiment, the components are disposable.

### BRIEF DESCRIPTION OF THE DRAWINGS

FIG. 1 schematically illustrates the components that may be used in the methods of the invention, wherein the test sample is fully immersed in the nucleic acid preservation solution.

FIG. 2 schematically illustrates the components that may be used in the methods of the invention, wherein the collection means of absorbent material has absorbed the majority of the nucleic acid preservation solution.

### DETAILED DESCRIPTION OF THE INVENTION

Embodiments of the present invention are described, by way of example only, with reference to the accompanying drawings. The size and shape of each component in the drawings is for illustrative purposes only and does not reflect or limit the size or shape of the components that may be used in the methods of invention in any way, except to the extent that each component should be functionally compatible with the other components used.

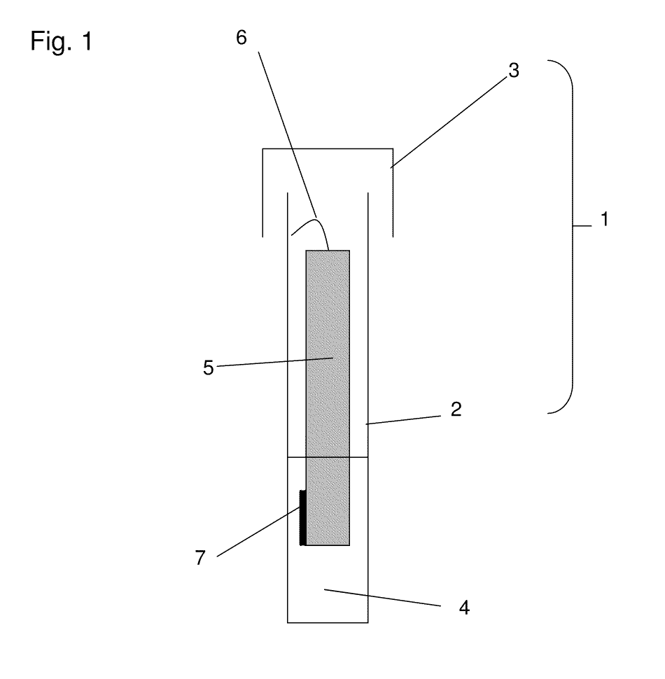

FIG. 1 illustrates the relative location of the components immediately after placement of the collection means of absorbent material (optionally carrying a test sample absorbed thereon) in the test sample receiving vessel. The components as shown in FIG. 1 comprise a test sample receiving vessel (1) formed of a rigid material, with a body portion (2) and (optionally) a lid portion (3). The body portion (2) accommodates both a volume of nucleic acid preservation solution (4) and a collection means of absorbent material (5). The collection means of absorbent material may optionally incorporate a removal cord or pull string (6). Preferably, the removal cord or pull string is also accommodated within the body portion of the test sample receiving vessel.

The test sample is also shown (7) as absorbed on the collection means of absorbent material (5). Different locations of the test sample (7) in relation to the collection means of absorbent material (5) are also possible. The test sample (7) is immersed in the nucleic acid preservation solution (4) in FIG. 1.

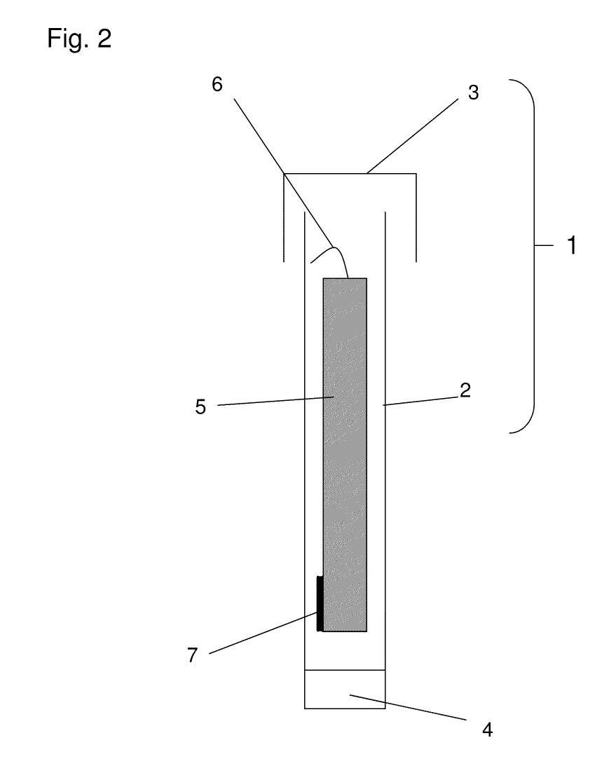

FIG. 2 illustrates a typical arrangement of the components that may be used in the methods of the invention some time after placement of the collection means of absorbent material (optionally carrying a test sample absorbed thereon) in the test sample receiving vessel and follows the labelling of FIG. 1. In the embodiment shown in FIG. 2, the collection means of absorbent material (5) has absorbed the majority of the nucleic acid preservation solution (4) that was present in the test sample receiving vessel (1).

### (i) Test Sample Receiving Vessel

The test sample receiving vessel (1) of the methods of the present invention is suitably sized to accommodate both a collection means of absorbent material (5), optionally carrying a test sample (7) absorbed thereon, and a nucleic acid preservation solution (4) in combination. Preferably, the test sample receiving vessel is optimally sized such that introduction of the collection means of absorbent material (5) into the test sample receiving vessel (1) in the presence of the nucleic acid preservation solution (4) results in partial or total immersion of the collection means of absorbent material (5) in the nucleic acid preservation solution (4). The collection means of absorbent material preferably absorbs a quantity of the nucleic acid preservation solution. Preferably, there is a sufficient amount of the nucleic acid preservation solution to allow saturation of the collection means of absorbent material. Most advantageously, the collection means of absorbent material absorbs the majority of the nucleic acid preservation solution, resulting in saturation of the collection means of absorbent material.

The test sample receiving vessel may comprise a body portion (2) and a lid portion (3). The body portion and lid portion in combination preferably create a sealable vessel which may (fully) accommodate both the nucleic acid preservation solution and the collection means of absorbent material. Preferably, the sealable vessel created by the body and lid portion of the test sample receiving vessel is optimally sized to accommodate the collection means of absorbent material, optionally carrying a test sample absorbed thereon, and approximately 10 to 30 ml of nucleic acid preservation solution. Advantageously, the sealable vessel can accommodate the collection means of absorbent material, optionally carrying a test sample absorbed thereon, and approximately 20 ml of nucleic acid preservation solution. The lid portion may have, but is not limited to having, a screw cap or a snap fit closure mechanism. Preferably, the screw cap or snap fit closure mechanism is childproof.

A suitable test sample receiving vessel may be made of glass, plastic, etc. or a combination of these materials. Preferably, the test sample receiving vessel is made of a material that is not degraded or affected by the contents of the nucleic acid preservation solution. For example, the material should not be degraded or affected by the presence of methanol in the nucleic acid preservation solution. Advantageously, the test sample receiving vessel is made of a plastic such as polypropylene. Preferably, the test sample receiving vessel is hygienically clean. In one embodiment, the test sample receiving vessel is made of low cost materials. In a further embodiment, the test sample receiving vessel is disposable. Disposable plastic culture bottles are particularly suitable. In particular, a childproof screw-capped 50 ml vial is preferable. However, the size, shape or construction of the receiving vessel is not material to the methods of the invention, provided that it is suitable for bringing a collection means of absorbent material (optionally carrying a test sample) into contact with a nucleic acid preservation solution.

### (ii) Nucleic Acid Preservation Solution

The methods of the present invention may employ a nucleic acid preservation solution. The nucleic acid preservation solution acts to maintain the integrity of nucleic acid molecule(s) within a sample to ensure that the sample is suitable for subsequent analysis. Preferably, the nucleic acid preservation solution minimises nucleic acid degradation. This may be achieved by inactivating DNase and/or RNase activity.

The nucleic acid preservation solution is preferably suitable for preserving nucleic acid at near ambient or ambient temperature. It is also suitable for long term storage at near ambient or ambient temperature in a sealed environment prior to collection of the sample. For example, the nucleic acid preservation solution may be stored at near ambient or ambient temperature in a sealed environment prior to collection of the sample for approximately at least one day, up to or at least one week, up to or at least one month, up to or at least six months, up to or at least one year, up to or at least two years or up to or at least five years. Preferably, the nucleic acid preservation solution is suitable for storage for up to or at least approximately two years. Preferably, the nucleic acid preservation solution is (relatively) temperature resistant and therefore, if required, it may be stored at a range of temperatures (for example between 4° C. or −20° C.). After collection of the sample, the sample may be retained in the nucleic acid preservation solution for a period of approximately at least one hour, up to or at least one day, up to or at least one week, up to or at least two weeks, up to or at least four weeks or up to or at least two months prior to analysis. Advantageously, the sample is retained in the nucleic acid preservation solution for up to or at least approximately four weeks prior to analysis.

In one embodiment, the nucleic acid preservation solution comprises methanol at a concentration in the range of at least 60% (v/v).

The nucleic acid preservation solution of the invention may comprise further components, provided that the required concentration of methanol is present. Such further components may include, for example, buffering agents, including but not limited to phosphate buffers, acetate buffers (e.g. sodium acetate, magnesium acetate or calcium acetate), Tris buffers, etc. Other components which may be present in the solution include chelating agents such as EDTA and salts thereof, particularly disodium, tripotassium or tetrasodium salts.

The invention provides a method of collecting one or more cells or tissues from the cervix of subject and preserving said cells or tissues in a manner that permits recovery of at least one nucleic acid molecule in a test sample. In the context of the invention, a ‘test sample’ is a sample that has been obtained from the cervix of a subject using a collection means of absorbent material. The test sample may be preserved and stored in a nucleic acid preservation solution as provided herein.

In one embodiment, a suitable volume of the nucleic acid preservation solution is provided to allow saturation of the collection means of absorbent material. Preferably, the collection means of absorbent material absorbs the majority of the nucleic acid preservation solution that is provided, resulting in saturation of the collection means of absorbent material. By way of example, approximately 20 ml of nucleic acid preservation solution may be provided in a 50 ml test sample receiving vessel, of which approximately 18 to 19 ml may be absorbed by the collection means of absorbent material, resulting in saturation of the collection means of absorbent material.

(iii) Collection Means of Absorbent Material

The collection means of absorbent material of the invention is used as a means for removing cervical cells from a subject. A suitable collection means of absorbent material may be, but is not limited to, a swab, a spatula with an absorbent surface or a disposable absorbent product of cotton and/or rayon staple wadding. Within the meaning of the invention ‘absorbent’ refers to a material that is suitable for absorbing liquid easily. In this context, materials such as plastic or wood are not ‘absorbent’. Preferably, the collection means of absorbent material is suitable for adsorbing a test sample from the cervix of a subject. Most preferably, after removal from the subject, the collection means of absorbent material is also suitable for absorbing a sufficient volume of nucleic acid preservation solution to result in preservation of the test sample.

The collection means of absorbent material must be suitable for insertion into the vaginal cavity of the subject. Preferably, the collection means of absorbent material is suitable for self-sampling. Most preferably, the collection means of absorbent material is suitable for insertion into the vaginal cavity by the subject herself, without the need for specialised equipment, skill or training. Optionally, the collection means of absorbent material further comprises a removal cord or pull string. Optionally, the collection means of absorbent material is a menstruation tampon. Preferably, the menstruation tampon further comprises an applicator. The applicator facilitates the insertion of the menstruation tampon into the vaginal cavity of the subject, whilst minimising contamination of the menstruation tampon surface by the subject. Advantages of a menstruation tampon include low cost, ease and familiarity of use by a subject and comfort. Ease and familiarity of use are especially important in the context of the invention. The ease and familiarity with which a menstruation tampon may be used allows women to collect and preserve a test sample from their own cervix without the need for intervention or help. By way of example, women should be able to insert the menstruation tampon in the correct location in the vaginal cavity easily. Insertion of the menstruation tampon can therefore take place within the comfort of the user's own home and/or at their own convenience. The use of a menstruation tampon for the collection of a test sample obtained from the cervix of a subject is highly advantageous in the context of the invention when compared to the spatulas and/or scrapers that are used in traditional cervical screening tests.

Preferably, the collection means of absorbent material is retained within the subject for a minimum of approximately one minute, at least ten minutes, at least thirty minutes, exactly one hour, or at least one hour to ensure that a suitable test sample is obtained. Preferably, the collection means of absorbent material is retained within the subject for a sufficient length of time to collect/adsorb a suitable test sample for subsequent mRNA extraction. In one embodiment, the collection means of absorbent material is retained within the subject for approximately one hour. In one embodiment, the collection means of absorbent material is made of a material that is relatively pliable. Accordingly, once the collection means of absorbent material is placed within the vaginal cavity of the subject, it can be retained in place relatively easily and comfortably.

The inventors have found that a menstruation tampon, inserted in the normal manner, can collect sufficient cervical cells to allow HPV testing based on nucleic acid (mRNA) amplification. Standard menstruation tampons that have been constructed in the conventional manner may be used. In addition, the method used to insert the menstruation tampon into the vaginal cavity can be equivalent to that used for insertion of a tampon during menstruation. In this embodiment of the invention, a menstruation tampon is used to collect the test sample without the need for ‘special’ techniques or materials.

In one embodiment of the invention, after the collection means of absorbent material is brought into contact with the nucleic acid preservation solution, a proportion of the nucleic acid preservation solution is absorbed by the collection means of absorbent material. In a preferred embodiment, the collection means of absorbent material absorbs the majority of the nucleic acid preservation solution that is provided. The collection means of absorbent material may absorb at least approximately 50%, 60%, 70%, 80%, 90% or 95% of the nucleic acid preservation solution. Preferably, a suitable volume of the nucleic acid preservation solution is provided to allow saturation of the collection means of absorbent material to occur. By way of example, approximately 20 ml of nucleic acid preservation solution may be provided in a 50 ml test sample receiving vessel, of which approximately 18 to 19 ml may be absorbed by the collection means of absorbent material, resulting in saturation of the collection means of absorbent material.

In a preferred embodiment, the collection means of absorbent material is hygienically clean prior to insertion into the vaginal cavity. Preferably, the collection means of absorbent material is made of low cost material(s). Most preferably, the collection means of absorbent material is disposable.

Optionally, the methods of the invention may further utilise additional components for facilitating the handling of the collection means of absorbent material. In one embodiment, gloves may be used. Preferably, the gloves are hygienically clean prior to use. The gloves may be used by the subject to minimise contamination of the collection means of absorbent material, test sample, nucleic acid preservation solution and/or test sample receiving vessel during the collection of the sample. Suitable gloves may be made of plastic or any other suitable material(s) such as, but not limited to, latex or rubber. Advantageously, the gloves are made of low cost material(s) and are, optionally, disposable.

### (v) A Method of the Invention

The present invention also provides methods for the collection and preservation of test samples obtained from a subject for subsequent analysis at, optionally, a remote location.

In one aspect of the invention, a method is provided that advantageously enables women to collect and preserve a test sample from their cervix for subsequent analysis at a site for analysis. The invention therefore provides individuals who do not wish to participate in a national cervical screening program access to cervical screening tests. Unwillingness to participate in a national screening program may be due to various ethical, personal or religious reasons. In one embodiment, the methods of the invention allow a test sample to be collected by a user without need for a trained medical practitioner. Accordingly, the test sample may be collected within the comfort of the user's own home.

In one aspect of the invention, a method of collecting one or more cells or tissues from the cervix of a subject and preserving said cells or tissues in a manner which permits recovery of at least one nucleic acid molecule in a test sample is provided. The method comprises, consists essentially of or consists of the steps of the subject contacting their cervix with a collection means of absorbent material to collect the test sample and bringing the test sample into contact with a nucleic acid preservation solution.

In one embodiment of this aspect, the collection means of absorbent material is inserted into the vaginal cavity of a subject. Insertion into the vaginal cavity is performed such that the collection means of absorbent material contacts the cervix of the subject and thereby obtains a test sample of one or more cervical cells or tissues. In one embodiment, the collection means of absorbent material is a menstruation tampon. The ease and familiarity with which a menstruation tampon may be used allows women to insert the menstruation tampon in the correct location in the vaginal cavity easily. Insertion of the menstruation tampon can therefore take place within the comfort of the user's own home and/or at their convenience. Preferably, the method used to insert the menstruation tampon into the vaginal cavity is equivalent to that used during insertion of a tampon during menstruation. In this embodiment of the invention, a menstruation tampon is inserted into the vaginal cavity without the need for ‘special’ techniques or methods.

Preferably, the collection means of absorbent material is retained within the subject for a minimum of approximately one minute, at least ten minutes, at least thirty minutes, exactly one hour, or at least one hour to ensure that a suitable test sample is obtained. Preferably, sufficient test sample is obtained to allow for subsequent RNA, preferably mRNA, extraction. In one embodiment, the collection means of absorbent material is retained within the subject for approximately one hour.

The collection means of absorbent material with the test sample is then removed from the subject and is brought into contact with a nucleic acid preservation solution. Preferably, the nucleic acid preservation solution is present within a test sample receiving vessel. In this preferred embodiment of the invention, the collection means of absorbent material with the test sample is brought into contact with a nucleic acid preservation solution by placement in the test sample receiving vessel containing the nucleic acid preservation solution.

The process of bringing the collection means of absorbent material into contact with a nucleic acid preservation solution may be facilitated by the use of gloves. Advantageously, the use of gloves minimises contamination of the test sample. In particular, gloves may minimise the transfer of RNase and/or DNase from the user to the test sample, collection means of absorbent material and/or the nucleic acid preservation solution. In one embodiment, the collection means of absorbent material further comprises a removal cord or pull string. The presence of a removal cord or pull string also reduces contamination of the test sample, collection means of absorbent material and/or the nucleic acid preservation solution. It facilitates removal of the collection means from the vaginal cavity and placement of the collection means of absorbent material in the nucleic acid preservation solution without the user needing to contact the area of the collection means of absorbent material that carries the test sample directly.

Optionally, the test sample receiving vessel used in the method comprises a lid portion and a body portion, wherein the lid portion fits the body portion to provide a sealable vessel which can (fully) accommodate the nucleic acid preservation solution and the collection means of absorbent material. In one embodiment of the invention, after the test sample (and optionally the collection means of absorbent material) is brought into contact with the nucleic acid preservation solution within a test sample receiving vessel, the body portion and lid portion of the test sample receiving vessel are combined to provide a sealed vessel. Preferably, the collection means of absorbent material then absorbs the majority of the nucleic acid preservation solution. The sealed vessel may then optionally be transported to a site for subsequent monitoring and/or analysis. In one embodiment of the invention, the steps of preservation and transportation of the test sample are performed at ambient or near ambient temperature.

As stated above, the methods of the invention provide a means for collecting one or more cells or tissues from the cervix of a subject and preserving said cells or tissues in a manner which permits recovery of at least one nucleic acid molecule in a test sample. In a preferred embodiment of the invention, the test sample is monitored and/or analysed using microbiological techniques. Preferably, DNA (genotype) or RNA analysis is performed. In a further preferred embodiment of the invention, the test sample is suitable for RNA extraction. Preferably, extracted mRNA is analysed. As a preferred example, the test sample may be analysed using Nucleic Acid Sequence Based Amplification (NASBA). For example, the test sample may be analysed for the presence of human papillomavirus (HPV) mRNA using NASBA. Such analysis may be performed using the PreTect™ HPV proofer assay.

In one embodiment, the test sample is suitable for subsequent detection of cervical malignancy in a subject. Preferably, the test sample is suitable for subsequent analysis of at least one nucleic acid molecule in the test sample. Optionally, the nucleic acid molecule comprises, consists essentially of or consists of RNA (preferably mRNA). In one embodiment, the test sample is analysed for the presence of human papillomavirus (HPV) mRNA. For example, the test sample may be analysed for the presence of E6/E7 mRNA. In one embodiment, the test sample is analysed for the presence of HPV mRNA derived from carcinogenic HPV types. Preferably, the presence of mRNA is detected using a multiplex Nucleic Acid Sequence Based Amplification (NASBA) assay. For example, the NASBA assay may exclusively detect HPV type specific E6/E7 mRNA from carcinogenic HPV types (for example the PreTect™ HPV proofer assay).

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all embodiments described herein are considered to be broadly applicable and combinable with any and all other consistent embodiments, as appropriate.

Various publications are cited herein, the disclosures of which are incorporated by reference in their entireties, particularly for the purposes cited herein.

### CLAUSES

1. A method of collecting one or more cells or tissues from the cervix of a subject and preserving said cells or tissues in a manner which permits recovery of at least one nucleic acid molecule in a test sample, the method comprising the steps of:

(i) the subject contacting their cervix with a collection means of absorbent material to collect the test sample; and
(ii) bringing the test sample into contact with a nucleic acid preservation solution.

2. The method according to clause 1, wherein the collection means of absorbent material is a disposable absorbent product of cotton and/or rayon staple wadding suitable for insertion into the vaginal cavity of the subject.

3. The method according to clause 1 or 2, wherein the collection means of absorbent material further comprises a removal cord or pull string.

4. The method according to any of clauses 1 to 3, wherein the collection means of absorbent material is a menstruation tampon.

5. The method according to clause 4, wherein the menstruation tampon further comprises an applicator.

6. The method according to any of clauses 1 to 5, wherein the collection means of absorbent material is retained within the vaginal cavity of the subject for a sufficient length of time to collect a test sample suitable for subsequent nucleic acid extraction.

7. The method according to clause 6, wherein the collection means of absorbent material is retained within the vaginal cavity of the subject for approximately at least one hour.

8. The method according to any of clauses 1 to 7, wherein step (ii) comprises placing the collection means of absorbent material into a test sample receiving vessel with a body portion.

9. The method according to clause 8, wherein the test sample receiving vessel further comprises a lid portion to fit the body portion, wherein the lid portion and body portion in combination provide a sealable vessel which can fully accommodate the nucleic acid preservation solution and the collection means of absorbent material.

10. The method according to clause 9, wherein the test sample receiving vessel is a 50 ml sealable container.

11. The method according to any of clauses 8 to 10, wherein the test sample receiving vessel contains a nucleic acid preservation solution.

12. The method according to any of clauses 1 to 11, wherein the test sample is immersed in the nucleic acid preservation solution during step (ii).

13. The method according to any of clauses 1 to 12, wherein the test sample is suitable for RNA extraction.

14. The method according to any of clauses 1 to 13, wherein the test sample is suitable for subsequent detection of a cervical malignancy in a subject.

15. The method of any of clauses 1 to 14, wherein the subject is an animal or human.

16. The method according to any of clauses 1 to 15, further comprising transporting the test sample from the site of collection from the subject to a site of clinical microbiological testing.

17. The method according to any of clauses 1 to 16, wherein the method is performed at ambient temperature.

18. The method according to clause 16, further comprising monitoring and/or analysis of the test sample at the site of clinical microbiological testing.

19. The method according to clause 18, wherein a time delay of more than one week occurs between bringing the test sample into contact with the nucleic acid preservation solution and the monitoring and/or analysis of the test sample.

20. The method according to clause 19, wherein the time delay is up to four weeks.

21. The method according to any of clauses 18 to 20, wherein the test sample is analysed using Nucleic Acid Sequence Based Amplification (NASBA).

22. The method according to any of clauses 1 to 21, wherein the nucleic acid molecule comprises RNA.

23. The method according to clause 22, wherein the RNA comprises mRNA.

24. The method according to clause 23, wherein the test sample is analysed for the presence of human papillomavirus (HPV) mRNA.

25. The method according to clause 24, wherein the test sample is analysed for the presence of E6/E7 mRNA.

26. The method according to clause 24 or 25, wherein the test sample is analysed for the presence of mRNA derived from carcinogenic HPV types.

27. The method according to clause 25 or 26, wherein the presence of HPV mRNA is detected using a multiplex Nucleic Acid Sequence Based Amplification (NASBA).

28. The method according to any of clauses 1 to 27, wherein the subject has cervical pre-cancer.

29. A method of collecting one or more cells or tissues from the cervix of a subject and preserving said cells or tissues in a manner which permits recovery of at least one nucleic acid molecule in a test sample, the method comprising the steps of:

(i) the subject contacting their cervix with a collection means of absorbent material to collect the test sample; and
(ii) bringing the test sample into contact with a nucleic acid preservation solution to preserve the test sample, wherein the preservation is performed at ambient temperature.

30. The method according to clause 29, wherein the collection means of absorbent material is a disposable absorbent product of cotton and/or rayon staple wadding suitable for insertion into the vaginal cavity of the subject.

31. The method according to clause 29 or 30, wherein the collection means of absorbent material further comprises a removal cord or pull string.

32. The method according to any of clauses 29 to 31, wherein the collection means of absorbent material is a menstruation tampon.

33. The method according to clause 32, wherein the menstruation tampon further comprises an applicator.

34. The method according to any of clauses 29 to 33, wherein the collection means of absorbent material is retained within the vaginal cavity of the subject for a sufficient length of time to collect a test sample suitable for subsequent nucleic acid extraction.

35. The method according to clause 34, wherein the collection means of absorbent material is retained within the vaginal cavity of the subject for approximately at least one hour.

36. The method according to any of clauses 29 to 35, wherein step (ii) comprises placing the collection means of absorbent material into a test sample receiving vessel with a body portion.

37. The method according to clause 36, wherein the test sample receiving vessel further comprises a lid portion to fit the body portion, wherein the lid portion and body portion in combination provide a sealable vessel which can fully accommodate the nucleic acid preservation solution and the collection means of absorbent material.

38. The method according to clause 37, wherein the test sample receiving vessel is a 50 ml sealable container.

39. The method according to any of clauses 36 to 38, wherein the test sample receiving vessel contains a nucleic acid preservation solution.

40. The method according to any of clauses 29 to 39, wherein the test sample is immersed in the nucleic acid preservation solution during step (ii).

41. The method according to any of clauses 29 to 40, wherein the test sample is suitable for RNA extraction.

42. The method according to any of clauses 29 to 41, wherein the test sample is suitable for subsequent detection of a cervical malignancy in a subject.

43. The method of any of clauses 29 to 42, wherein the subject has cervical pre-cancer.

44. The method of any of clauses 29 to 43, wherein the subject is an animal or human.

45. The method according to any of clauses 29 to 44, further comprising transporting the test sample at ambient temperature from the site of collection from the subject to a site of clinical microbiological testing.

46. The method according to clause 45, further comprising monitoring and/or analysis of the test sample at the site of clinical microbiological testing.

47. The method according to clause 46, wherein a time delay of more than one week occurs between bringing the test sample into contact with the nucleic acid preservation solution and the monitoring and/or analysis of the test sample.

48. The method according to clause 47, wherein the time delay is up to four weeks.

49. The method according to any of clauses 46 to 48, wherein the test sample is analysed using Nucleic Acid Sequence Based Amplification (NASBA).

50. The method according to any of clauses 29 to 49, wherein the nucleic acid molecule comprises RNA.

51. The method according to clause 50, wherein the RNA comprises mRNA.

52. The method according to clause 51, wherein the test sample is analysed for the presence of human papillomavirus (HPV) mRNA.

53. The method according to clause 52, wherein the test sample is analysed for the presence of E6/E7 mRNA.

54. The method according to clause 52 or 53, wherein the test sample is analysed for the presence of mRNA derived from carcinogenic HPV types.

55. The method according to clause 53 or 54, wherein the presence of HPV mRNA is detected using a multiplex Nucleic Acid Sequence Based Amplification (NASBA).

56. A method of detecting cervical pre-cancer, a pre-disposition to cervical cancer or an incidence of cervical malignancy in a subject, the method comprising:

(i) collecting one or more cells or tissues from the cervix of a subject;
(ii) preserving said one or more cells or tissues;
(iii) transporting the test sample from the site of collection from the subject to a site of clinical microbiological testing; and
(iv) monitoring and/or analysing the test sample at the site of clinical microbiological testing in order to inform the subject that they have cervical pre-cancer, a pre-disposition to cervical cancer or an incidence of cervical malignancy.

57. The method according to clause 56, wherein steps (i), (ii), (iii) and/or (iv) are performed at ambient temperature.

58. A method of detecting cervical pre-cancer, a pre-disposition to cervical cancer or an incidence of cervical malignancy in a subject, the method comprising:

(i) supplying the subject with a sampling kit suitable for the collection of one or more cells or tissues from their cervix;
(ii) the subject collecting one or more cells or tissues from their cervix and sending the test sample to a site of clinical microbiological testing;
(iii) monitoring and/or analysing the test sample at the site of clinical microbiological testing in order to detect cervical pre-cancer, a pre-disposition to cervical cancer or an incidence of cervical malignancy; and,
(iv) transmitting the result of step (iii) to the subject or a healthcare professional.

59. The method according to clause 58, wherein test sample is sent to a site of clinical microbiological testing at ambient temperature.

60. The method of clause 58 or 59, wherein step (iv) comprises transmitting the result by email, telephone, facsimile or text message.

61. The method according to any of the preceding clauses, wherein the subject has no previous signs of cervical cancer.

62. A method of detecting cervical pre-cancer in an asymptomatic subject, the method comprising:

(i) collecting one or more cells or tissues from the cervix of a subject;
(ii) preserving said one or more cells or tissues;
(iii) transporting the test sample from the site of collection from the subject to a site of clinical microbiological testing; and
(iv) detecting the presence of E6/E7 mRNA derived from one or more carcinogenic HPV types.

63. The method according to clause 62, wherein steps (i), (ii), (iii) and/or (iv) are performed at ambient temperature.

64. A method of detecting cervical pre-cancer in an asymptomatic subject, the method comprising:

(i) supplying the subject with a self-sampling kit suitable for the collection of one or more cells or tissues from their cervix;
(ii) the subject collecting one or more cells or tissues from their cervix and sending the test sample to a remote site of clinical microbiological testing;
(iii) monitoring and/or analysing the test sample at the site of clinical microbiological testing in order to detect the presence of E6/E7 mRNA derived from one or more carcinogenic HPV types; and,
(iv) transmitting the result of step (iii) to the subject or a healthcare professional.

65. The method according to clause 64, wherein test sample is sent to a site of clinical microbiological testing at ambient temperature.

66. The method of clause 64 or 65, wherein step (iv) comprises transmitting the result by email, telephone, facsimile or text message.

We claim:

1. A method of collecting one or more cells or tissues from the cervix of a subject and preserving said cells or tissues in a manner which permits recovery of at least one nucleic acid molecule in a test sample, the method comprising the steps of:
(i) the subject contacting their cervix with a collection means of absorbent material to collect the test sample; and
(ii) bringing the test sample into contact with a nucleic acid preservation solution.

2. The method according to claim 1, wherein the collection means of absorbent material is a menstruation tampon.

3. The method according to claim 1, wherein the test sample is suitable for RNA extraction.

4. The method according to claim 1, wherein the test sample is suitable for subsequent detection of a cervical malignancy in a subject.

5. The method according to claim 1, further comprising transporting the test sample from the site of collection from the subject to a site of clinical microbiological testing where the test sample is monitored and/or analysed.

6. The method according to claim 1, wherein the method is performed at ambient temperature.

7. The method according to claim 5, wherein a time delay of more than one week occurs between bringing the test sample into contact with the nucleic acid preservation solution and the monitoring and/or analysis of the test sample.

8. The method according to claim 5, wherein the test sample is analysed for the presence of human papillomavirus (HPV) mRNA.

9. The method according to claim 8, wherein the presence of HPV mRNA is detected using a multiplex Nucleic Acid Sequence Based Amplification (NASBA).

10. The method according to claim 1, wherein the subject has cervical pre-cancer.

11. A method of collecting one or more cells or tissues from the cervix of a subject and preserving said cells or tissues in a manner which permits recovery of at least one nucleic acid molecule in a test sample, the method comprising the steps of:
(i) the subject contacting their cervix with a collection means of absorbent material to collect the test sample; and
(ii) bringing the test sample into contact with a nucleic acid preservation solution to preserve the test sample, wherein the preservation is performed at ambient temperature.

12. The method according to claim 11, wherein the collection means of absorbent material is a menstruation tampon.

13. The method according to claim 11, wherein the test sample is suitable for RNA extraction.

14. The method according to claim 11, wherein the test sample is suitable for subsequent detection of a cervical malignancy in a subject.

15. The method according to claim 11, wherein the subject has cervical pre-cancer.

16. The method according to claim 11, further comprising transporting the test sample at ambient temperature from the site of collection from the subject to a site of clinical microbiological testing where the test sample is monitored and/or analysed.

17. The method according to claim 16, wherein a time delay of more than one week occurs between bringing the test sample into contact with the nucleic acid preservation solution and the monitoring and/or analysis of the test sample.

18. The method according to claim 16, wherein the test sample is analysed for the presence of human papillomavirus (HPV) mRNA.

19. The method according to claim 18, wherein the presence of HPV mRNA is detected using a multiplex Nucleic Acid Sequence Based Amplification (NASBA).

20. A method of detecting cervical pre-cancer, a pre-disposition to cervical cancer or an incidence of cervical malignancy in a subject, the method comprising:
(i) collecting one or more cells or tissues from the cervix of a subject;
(ii) preserving said one or more cells or tissues;
(iii) transporting the test sample from the site of collection from the subject to a site of clinical microbiological testing; and
(iv) monitoring and/or analysing the test sample at the site of clinical microbiological testing in order to detect cervical pre-cancer, a pre-disposition to cervical cancer or an incidence of cervical malignancy.

21. A method of detecting cervical pre-cancer, a pre-disposition to cervical cancer or an incidence of cervical malignancy in a subject, the method comprising:
(i) supplying the subject with a sampling kit suitable for the collection of one or more cells or tissues from their cervix;
(ii) the subject collecting one or more cells or tissues from their cervix and sending the test sample to a site of clinical microbiological testing;
(iii) monitoring and/or analysing the test sample at the site of clinical microbiological testing in order to detect cervical pre-cancer, a pre-disposition to cervical cancer or an incidence of cervical malignancy; and
(iv) transmitting the result of step (iii) to the subject or a healthcare professional.
